(19) United States
(12) Patent Application Publication
(10) Pub. No.: US 2014/0066970 A1
(43) Pub. Date: Mar. 6, 2014

(54) ENDOVASCULAR MEDICAL SYSTEM INCLUDING EXPANDABLE AND COLLAPSIBLE FRAMEWORK AND METHOD USING SAME

(71) Applicant: Cook Medical Technologies LLC, Bloomington, IN (US)

(72) Inventor: Shavonna Warren, Bloomington, IN (US)

(73) Assignee: Cook Medical Technologies LLC, Bloomington, IN (US)

(21) Appl. No.: 13/936,255

(22) Filed: Jul. 8, 2013

Related U.S. Application Data
(60) Provisional application No. 61/694,803, filed on Aug. 30, 2012.

(51) Int. Cl. A61B 17/12

(52) U.S. Cl. CPC A61B 17/12172
USPC 606/200

(57) ABSTRACT

An endovascular medical system includes an expandable and collapsible framework including a plurality of link sets joined together at hubs. Each of the link sets includes a plurality of scissors linkages connected in series. The framework is movable between an expanded position in which each of the scissors linkages are pivoted to a closed position and a collapsed position in which each of the scissors linkages are pivoted to an open position. The endovascular medical system also includes a deployment system for advancing the framework to a vascular deployment site within a vascular structure. The deployment system restricts movement of the framework from the collapsed position to the expanded position during advancement to the vascular deployment site.

### RELATION TO OTHER PATENT APPLICATION

This application claims priority to provisional patent application 61/694,803, filed on Aug. 30, 2012, with the same title.

### TECHNICAL FIELD

The present disclosure relates generally to an endovascular medical system including an expandable and collapsible framework, and more particularly to a framework including a series of scissors linkages forming each of a plurality of interconnected link sets.

### BACKGROUND

Embolization procedures are designed to create an artificial blockage, or occlusion, within a vessel to block blood from flowing downstream from the blockage. These procedures are used to treat several conditions, including, for example, aneurysms, hemorrhages, and lesions or growths. Specifically, for example, an embolic device may be used to occlude blood flow to an aneurysm and, thus, reduce the risk of the aneurysm rupturing and producing internal hemorrhaging. An embolization procedure may also be used to isolate a treatment area from general circulation. In particular, for example, if a toxic agent, such as a chemotherapeutic agent, is delivered to a specific treatment site, it may be desirable to prevent circulation of the toxic agent downstream from the treatment area. As such, one or more artificial blockages may be created to effectively isolate the treatment area. Embolic devices may include physical barriers, such as coils, balloons, chemicals, and the like.

An exemplary embolic device is taught in U.S. Pat. No. 6,802,851 to Jones et al. (hereinafter Jones). In particular, Jones teaches a method and device for treating an aneurysm of a patient. The device includes a cup-shaped framework for supporting one or more embolization elements that is introduced into the aneurysm of the patient. A stent, which is connected to the cup-shaped framework, is compressed against an inner wall of the patient vessel to anchor the framework. Although Jones may present one solution for artificially occluding an aneurysm, there is a continuing need for endovascular medical devices that are reliable and effective. Further, there is a continuing need for medical devices that may be useful in a broad range of endovascular procedures.

The present disclosure is directed toward one or more of the problems or issues set forth above.

### SUMMARY OF THE DISCLOSURE

In one aspect, an endovascular medical system includes an expandable and collapsible framework including a plurality of link sets joined together at hubs. Each of the link sets includes a plurality of scissors linkages connected in series. The expandable and collapsible framework is movable between an expanded position in which each of the scissors linkages are pivoted to a closed position, and a collapsed position in which each of the scissors linkages are pivoted to an open position. The endovascular medical system also includes a deployment system for advancing the expandable and collapsible framework to a vascular deployment site within a vascular structure. The deployment system restricts movement of the expandable and collapsible framework from the collapsed position to the expanded position during advancement to the vascular deployment site.

In another aspect, a method of performing a percutaneous endovascular procedure using an endovascular medical system is provided. The endovascular medical system includes an expandable and collapsible framework including a plurality of link sets joined together at hubs. Each of the link sets includes a plurality of scissors linkages connected in series. The expandable and collapsible framework is movable between an expanded position in which each of the scissors linkages are pivoted to a closed position, and a collapsed position in which each of the scissors linkages are pivoted to an open position. The method includes steps of advancing the expandable and collapsible framework to a vascular deployment site within a vascular structure in the collapsed position, including restricting movement of the expandable and collapsible framework from the collapsed position to the expanded position using a deployment system. The deployment system is reconfigured to allow movement of the expandable and collapsible framework from the collapsed position to the expanded position. The framework is then moved from the collapsed position to the expanded position at the vascular deployment site by pivoting each of the scissors linkages from an open position to a closed position.

### BRIEF DESCRIPTION OF THE DRAWINGS

FIG. 1 is a side diagrammatic view of an endovascular medical system, according to one embodiment of the present disclosure;

FIG. 2 is a perspective view of an expandable and collapsible framework of the endovascular medical system of FIG. 1, shown in a collapsed position;

FIG. 3 is a perspective view of the expandable and collapsible framework of FIG. 2, shown in an expanded position;

FIG. 4 is a perspective view of a series of scissors linkages defining a link set of the expandable and collapsible framework of FIGS. 1-3, shown in a collapsed position;

FIG. 5 is a perspective view of the link set of FIG. 4, shown in an expanded position;

FIG. 6 is a side diagrammatic view of a vascular structure of a patient at one stage of an exemplary percutaneous endovascular procedure;

FIG. 7 is a side diagrammatic view of the vascular structure at another stage of the percutaneous endovascular procedure, shown with the expandable and collapsible framework in the collapsed position;

FIG. 8 is a side diagrammatic view of the vascular structure at another stage of the percutaneous endovascular procedure, shown with the expandable and collapsible framework self-expanded into the expanded position;

FIG. 9 is a side diagrammatic view of the vascular structure at one stage of an alternative percutaneous endovascular procedure, shown with the expandable and collapsible framework expanded using an inflatable balloon;

FIG. 10 is a side diagrammatic view of the vascular structure at another stage of the percutaneous endovascular procedure, shown with the expandable and collapsible framework detached from a deployment system and including a flow restriction membrane supported on the expandable and collapsible framework;

FIG. 11 is a side diagrammatic view of the vascular structure at one stage of an alternative percutaneous endovascular procedure, shown with an embolic device delivery catheter for inserting embolic devices within the expanded framework;

FIG. 12 is a side diagrammatic view of the vascular structure having the expandable and collapsible framework, in an expanded position, positioned therein;

FIG. 13 is a side diagrammatic view of the vascular structure having a semispherical-shaped expandable and collapsible framework, in an expanded position, positioned therein; and

FIG. 14 is a side diagrammatic view of the vascular structure at another stage of the percutaneous endovascular procedure, shown with the deployment system retracting the expandable and collapsible framework.

### DETAILED DESCRIPTION

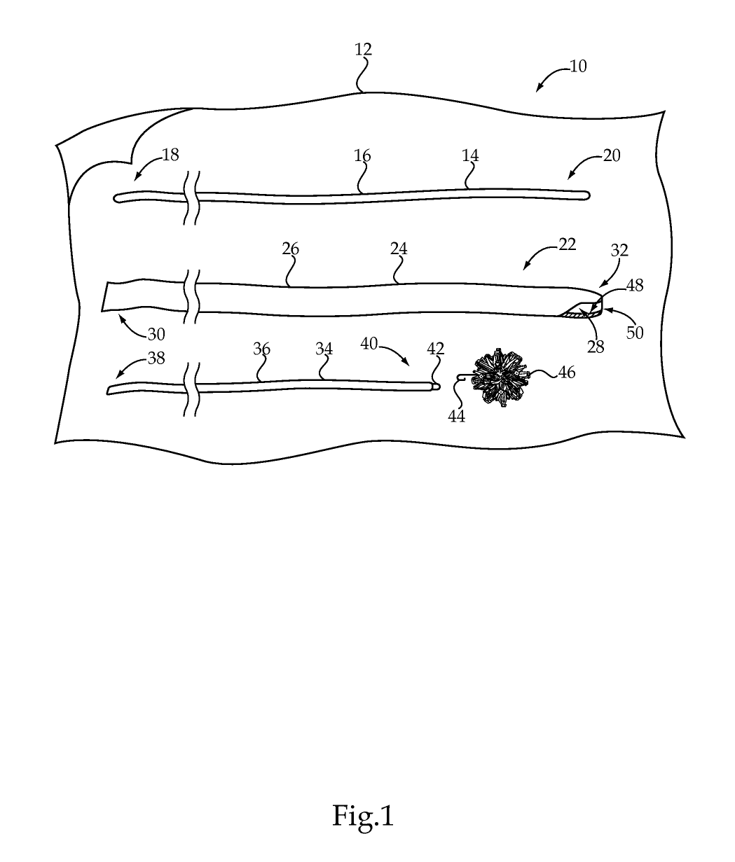

Referring to FIG. 1, there is shown an endovascular medical system 10, according to one embodiment of the present disclosure. The endovascular medical system 10 may include a number of components, which may be provided within a sterile, tear open package 12, as is known in the art. In performing a percutaneous endovascular medical procedure, some or all of the components of the endovascular medical system 10 may be used, depending upon the specifics of the procedure to be performed. As should be appreciated, however, the components shown in FIG. 1 might be separately packaged and/or the endovascular medical system 10 might also include components in addition to those shown, including components routinely used in percutaneous endovascular medical procedures.

The endovascular medical system 10 may include at least one wire guide 14, which is a device commonly used in percutaneous medical procedures to introduce a wide variety of medical devices into a vascular structure of a patient. Generally speaking, wire guide 14 includes an elongate flexible body 16 extending from a proximal end 18 to a distal end 20. Since wire guides are known, wire guide 14 will not be discussed herein in greater detail. However, it should be noted that wire guide 14 may be made from any of a number of known materials commonly used to manufacture medical devices and may include any of a variety of known configurations. For example, some wire guides include an elongate core element with one or more tapered sections near a distal end thereof. In the present disclosure, “proximal” will be used to refer to the end of a component or feature that is closest to a clinician, while “distal” is used to refer to a component or feature that is farthest away from the clinician. Such meanings are consistent with conventional use of the terms and, as such, should be understood by those skilled in the art.

A deployment system 22, which may include any number of components, may also be provided with the endovascular medical system 10. As shown in the exemplary embodiment, the endovascular medical system 10 may include a deployment catheter 24, or sheath, which may function as a deployment device for the endovascular medical system 10. The deployment catheter 24 generally includes an elongate tubular body 26 defining a lumen 28 extending from an open proximal end 30 to an open distal end 32 of the elongate tubular body 26. The elongate tubular body 26, which may be distally tapered, may be made from any common medical tube material, such as, for example, a plastic, rubber, silicone, or Teflon® material, and may exhibit both stiffness, or firmness, and flexibility. Materials as well as dimensions may vary depending on the particular application.

The endovascular medical system 10 may also include a deployment wire 34, also referred to as a pusher wire or retraction wire, which, together with the deployment catheter 24, may define the deployment system 22 of the endovascular medical system 10. The deployment wire 34 may generally include an elongate flexible body 36 extending from a proximal end 38 to a distal end 40 and may be similar to the wire guide 14 in materials and/or dimensions. It should be appreciated that the wire guide 14, deployment catheter 24, and deployment wire 34 may all range in length from several inches to several feet long, and may all have wall diameters that are orders of magnitude smaller than their lengths. The deployment wire 34 may also include a retraction member 42, which may include an open or closed loop or hook, shaped to engage a retraction hook 44 of an expandable and collapsible framework 46 of the endovascular medical system 10.

The expandable and collapsible framework 46, which will be discussed in greater detail below, may be introduced into a vascular structure of a patient using the deployment system 22 described herein, or using an alternative deployment system or device. For example, the retraction member 42 of the deployment wire 34 may be engaged with the retraction hook 44 of the expandable and collapsible framework 46 to define an engaged configuration. In the engaged configuration, the deployment wire 34 and the expandable and collapsible framework 46 may be advanced together through the lumen 28 of the deployment catheter 24, with inner walls 48 defining the catheter lumen 28 restricting radial movement, or expansion, of the expandable and collapsible framework 46 during the advancement. As should be appreciated, the deployment wire 34 may require a stiffness sufficient for advancing and/or retracting the expandable and collapsible framework 46 through the deployment catheter 24. Although a hook and loop type engagement is shown, it should be appreciated that any type of releasable connection between the deployment wire 34 and the expandable and collapsible framework 46 may be used.

Although not shown, a handle or retraction mechanism may be provided at the proximal end 30, 38 of one or both of the deployment catheter 24 and the deployment wire 34 to aid in the deployment and/or retraction of the expandable and collapsible framework 46. In particular, for example, a handle may be provided to facilitate movement of the deployment wire 34, and the expandable and collapsible framework 46, relative to the deployment catheter 24. Such movement allows the expandable and collapsible framework 46 to be positioned distally beyond a distal tip 50 of the deployment catheter 24 such that the framework 46 is no longer restricted from radial movement, or expansion, by the catheter lumen walls 48. Although a particular deployment system 22 is shown, it should be appreciated that any deployment system or device capable of advancing the expandable and collapsible framework 46 through a vascular structure in a collapsed position and allowing movement of the framework 46 into an expanded position at a vascular deployment site is contemplated.

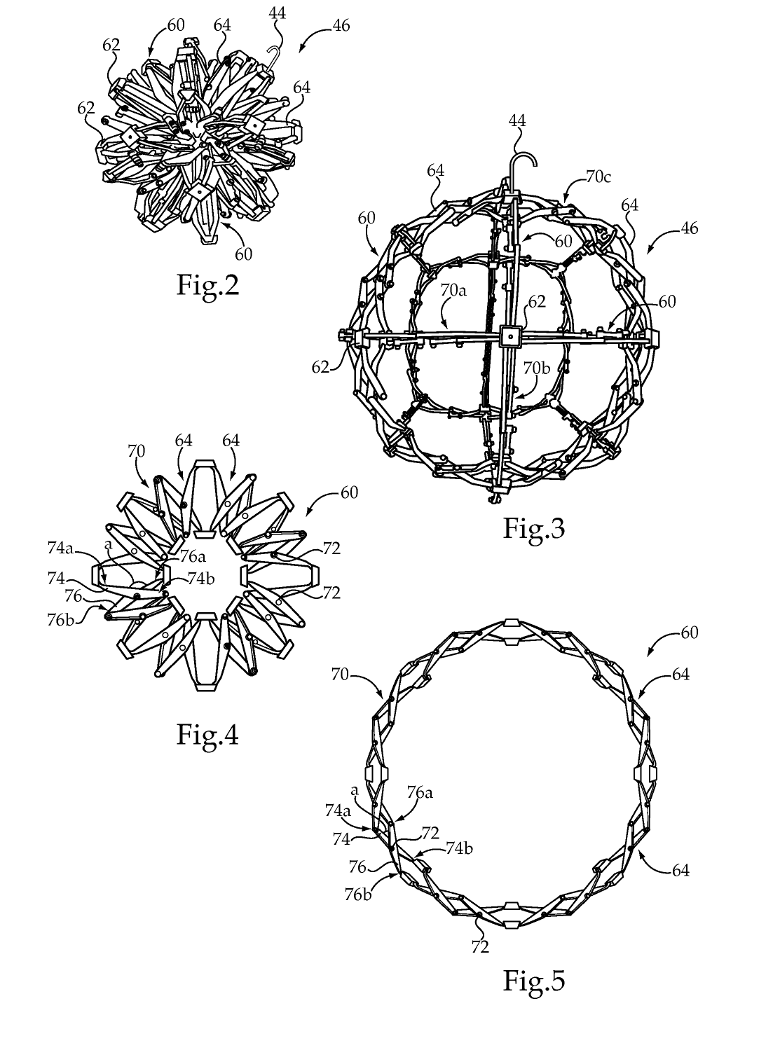

As shown in FIGS. 2 and 3, the expandable and collapsible framework 46 includes a plurality of link sets 60 joined together at hubs 62. Each of the link sets 60 includes a plurality of scissors linkages 64 connected in series. The expandable and collapsible framework 46 is movable between a collapsed position, shown in FIG. 2, in which each of the scissors linkages 64 are pivoted to an open position, and an expanded position, shown in FIG. 3, in which each of the scissors linkages 64 are pivoted to a closed position. As shown, the expandable and collapsible framework 46 may have a spherical shape in both of the collapsed and expanded positions, with the framework 46 having an increased diameter in the expanded position. To be clear, outer points of the expandable and collapsible framework 46 may define a spherical shape, with the diameter of the spherical shape at least doubling when the framework 46 is moved from the collapsed position to the expanded position.

Referring also to FIGS. 4 and 5, a portion of the link sets 60 may define a continuous ring, or circle, 70. The continuous ring 70 is collapsed, as shown in FIG. 4, when each of the scissors linkages 64 is pivoted about a respective pivot axis 72 toward an open position, and is expanded, as shown in FIG. 5, when each of the scissors linkages 64 is pivoted toward a closed position. In particular, each of the scissors linkages 64 includes links 74 and 76. The links 74 and 76 have opposing ends 74a and 74b and opposing ends 76a and 76b, respectively, and are joined together at the pivot axis, or pivot joint, 72. The pivot joint 72 of the links 74 and 76, which may be non-linear or curved, may be offset with respect to a center of the links 74 and 76, depending on the position of the scissors linkage 64. To form the continuous ring 70, ends 74a and 76a are connected, such as at pivot joints, with opposing ends 74a and 74b of a sequential scissors linkage 64. The scissors linkages 64 are pivoted toward an open position when ends 74a and 76a and ends 74b and 76b are moved away from one another. Alternatively, the scissors linkages 64 are pivoted toward a closed position when ends 74a and 76a and ends 74b and 76b are moved toward one another, as shown in FIG. 5.

As used herein, a closed position of the scissors linkages 64 may include positions in which an angle a defined by the links 74 and 76, as called out in FIGS. 4 and 5, is about 90 degrees or less. An open position of the scissors linkages 64 may include positions in which the angle a is about 90 degrees or greater. As should be appreciated, the collapsed position of the continuous ring 70, as shown in FIG. 4, corresponds to the collapsed position of the expandable and collapsible framework 46 shown in FIG. 2. The expanded position of the continuous ring 70, as shown in FIG. 5, corresponds to the expanded position of the expandable and collapsible framework, as shown in FIG. 5.

As shown, the expandable and collapsible sphere 46 may include at least three continuous rings 70. In particular, the expandable and collapsible sphere 46 may include a first continuous ring 70a that lies in a plane parallel to the x-axis, a second continuous ring 70b that lies in a plane parallel to the y-axis, and a third continuous ring 70c that lies in a plane parallel to the z-axis. In particular, each of the continuous rings 70 may lie in a plane that interests a center of the spherical shaped expandable and collapsible framework 46. Each of the continuous rings 70 may be interconnected at the hubs 62 referenced above. In particular, a hub 62 may include a location at which more than two sets of scissors linkages 64 are connected. As shown, additional link sets 60, which may not define continuous rings 70, may also be provided, and may be interconnected with the continuous rings 70 at hubs 62.

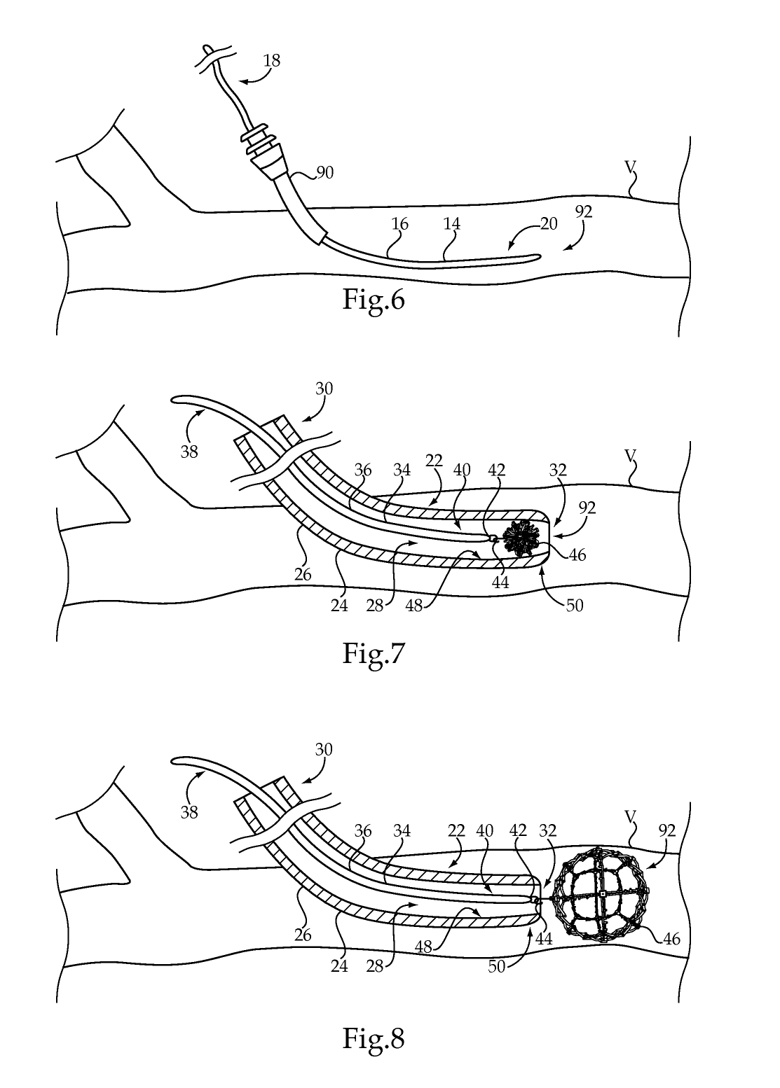

Referring to FIG. 6, there is shown a vascular structure V of a patient having a needle 90, or introducer, positioned therein, at a first stage of an exemplary percutaneous endovascular procedure. The procedure, further described herein, may include a vascular occlusion procedure, a stent placement procedure, or a vascular filter placement procedure, to name a few. At a next stage of the procedure, a clinician may insert the wire guide 14 through a tube of the needle 90 and into the vascular structure V such that the distal end 20 of the wire guide 14 is at or near a vascular deployment site 92. After the wire guide 14 is properly positioned, the needle 90 may be removed and the deployment system 22, or components thereof, may be inserted over the wire guide 14, as is known in the art. Thereafter, the wire guide 14 may be removed.

As shown in FIG. 7, the expandable and collapsible framework 46 may be advanced to the vascular deployment site 92 within the vascular structure V in the collapsed position of FIG. 2. In particular, the catheter lumen walls 48 may restrict movement of the expandable and collapsible framework 46 from the collapsed position to the expanded position. The deployment system 22 may then be reconfigured to allow movement of the expandable and collapsible framework 46 from the collapsed position to the expanded position. Specifically, for example, one or both of the deployment catheter 24 and the deployment wire 34 may be moved relative to the other such that the expandable and collapsible framework 46 is no longer restricted form radial movement by the catheter lumen walls 48. As such, the expandable and collapsible framework 46 may be moved from the collapsed position (FIG. 2) to the expanded position (FIG. 3), as shown in FIG. 8. In particular, each of the scissors linkages 64 may be pivoted from the open position to the closed position, as described above.

According to some embodiments, the expandable and collapsible framework 46 may be self-expanding from the collapsed position to the expanded position. As such, the expandable and collapsible framework 46 may be made from a resilient or shape memory material, such as, for example, nitinol, that is capable of self-expanding from the collapsed position to the expanded position. In particular, the expandable and collapsible framework 46 may be restricted from self-expansion using the deployment catheter 24. Once the deployment catheter 24 is advanced to the vascular deployment site 92, the deployment system 22 may be reconfigured such that the expandable and collapsible framework 46 is no longer restricted from radial expansion by the deployment catheter 24. As a result, the expandable and collapsible framework 46 self-expands to an expanded diameter. As should be appreciated, the expandable and collapsible framework 46 may be selected to provide the desired expanded diameter for a particular application or procedure.

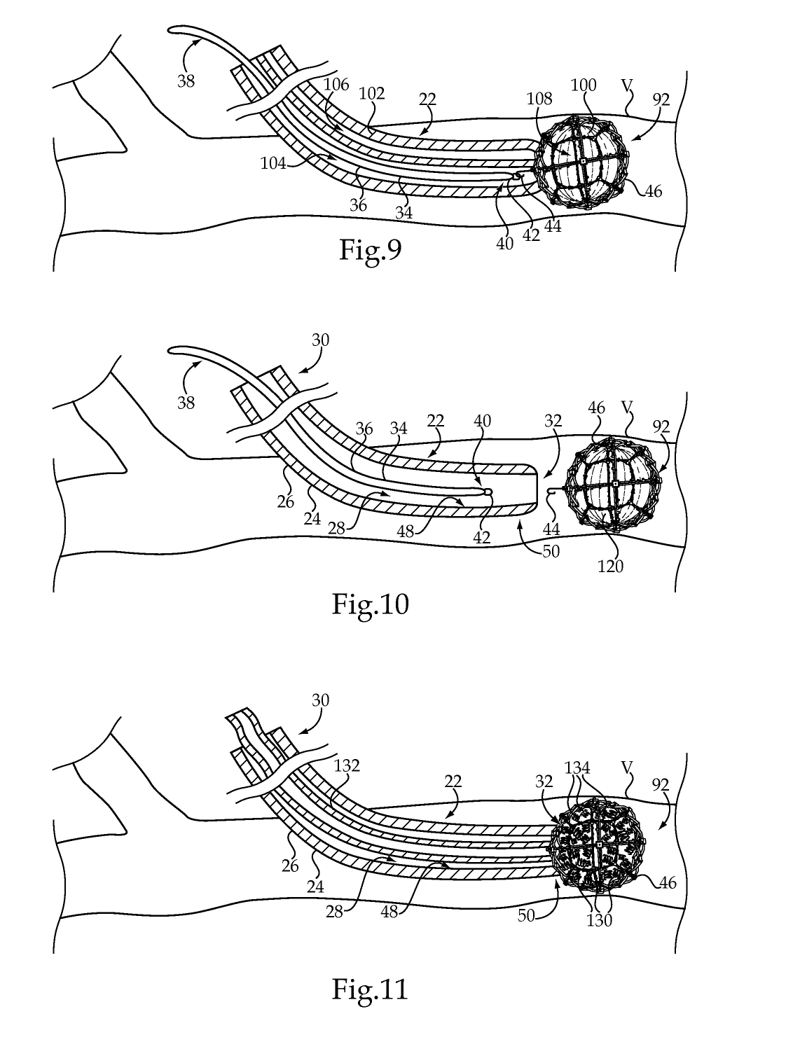

Alternatively, as shown in FIG. 9, an inflatable balloon 100 may be positioned within the expandable and collapsible framework 46 such that the inflatable balloon 100 may be inflated to move the framework 46 from the collapsed position to the expanded position. According to such an embodiment, a deployment catheter 102 may be used that includes a first lumen 104 for deployment of the expandable and collapsible framework 46 via the deployment wire 34, and a second lumen 106 fluidly connected with an interior 108 of the inflatable balloon 100 and defining an inflation lumen 106 for the inflatable balloon 100, as is known in the art. According to this exemplary embodiment, the expandable and collapsible framework 46 may remain attached to the deployment catheter 102 and/or the deployment wire 34 during the procedure.

According to other embodiments, it may be desirable to detach the expandable and collapsible framework 46 from the deployment catheter 24, or deployment catheter 102, and the deployment wire 34. For example, as shown in FIG. 10, the deployment wire 34 may be detached from the expandable and collapsible framework 46 by disengaging the retraction member 42 of the deployment wire 34 from the refraction hook 44 of the framework 46. The deployment catheter 24 and deployment wire 34 may then be withdrawn from the vascular structure V, leaving the expandable and collapsible framework 46 in place. According to a particular use, and as shown in FIG. 10, a flow restriction membrane 120 may be supported on the expandable and collapsible framework 46 and, thus, may allow the expanded framework 46 to act as an occlusion device within the vascular structure V. The flow restriction membrane 120 may include a biocompatible material capable of reducing or restricting blood flow.

The expandable and collapsible framework 46 may also provide artificial occlusion using embolic devices 130 positioned within the framework 46. For example, as shown in FIG. 11, an embolic device delivery catheter 132 may be inserted through the deployment catheter 24 and used to delivery a plurality of embolic devices 130, such as embolic coils 134 or other occlusive material, into the expandable and collapsible framework 46 in a known manner. After delivery of the embolic devices 130 into the expandable and collapsible framework 46, one or both of the catheters 24 and 132 may be removed from the vascular structure V.

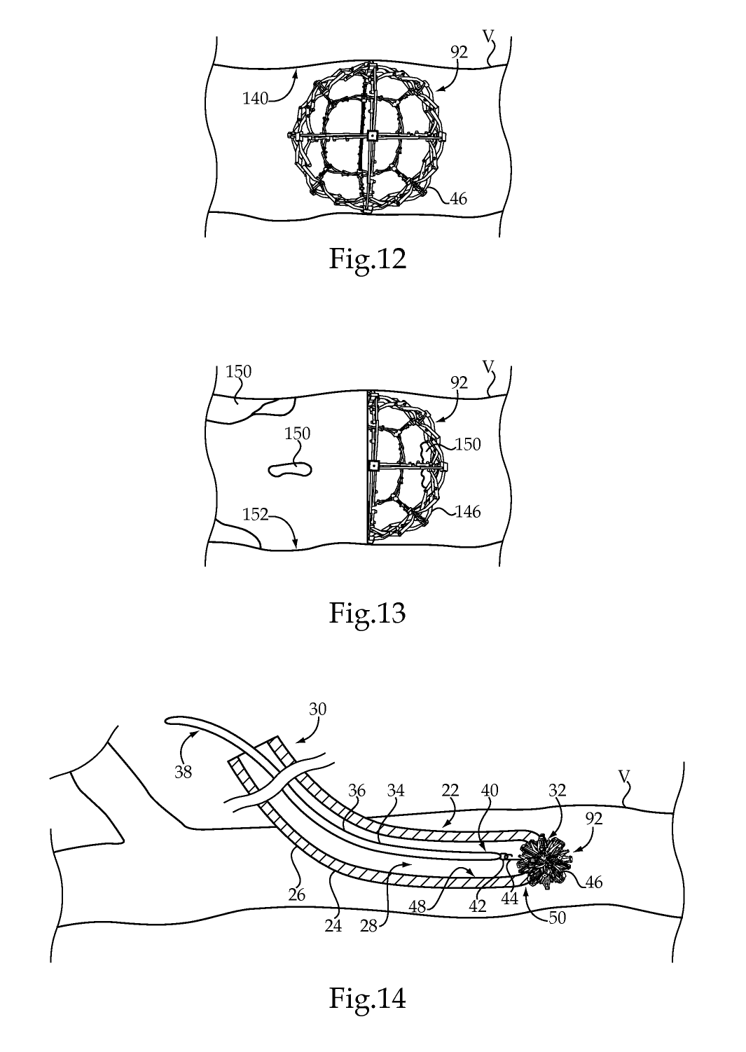

According to additional uses, and as shown in FIG. 12, the expandable and collapsible framework 46 may be used as a stent to exert a radial force against walls 140 of the vascular structure V. An alternative expandable and collapsible framework 146 may be provided in a semispherical shape, as shown in FIG. 13, and, when expanded within the vascular structure V, may act as a filter. For example, the expandable and collapsible framework 146 may collect clots 150, such as clots 150 dislodged from walls 152 of the vascular structure V.

When desired, the expandable and collapsible framework 46 may be withdrawn from the vascular structure V by engaging the retraction member 42 of the deployment wire 34 with the retraction hook 44 of the framework 46. While in the engaged configuration, the deployment wire 34, or retraction device, and the expandable and collapsible framework 46 may be withdrawn from the deployment catheter 24 and, thus, the vascular structure V. While retracting the expandable and retractable framework 46 into the deployment catheter 24, the catheter lumen walls 48 will exert a force against the expandable and collapsible framework 46 and move the framework 46 from the expanded configuration to the collapsed configuration.

### INDUSTRIAL APPLICABILITY

The present disclosure is generally applicable to endovascular medical systems and devices. More specifically, the present disclosure is applicable to endovascular medical devices reliable and effective for use in a variety of different percutaneous endovascular procedures. Yet further, the present disclosure is applicable to an endovascular medical device that may be used for a variety of purposes, including, for example, vascular occlusion, vascular wall support or repair, and vascular filtration.

Referring generally to FIGS. 1-14, an endovascular medical system 10 may generally include an expandable and collapsible framework 46, and a deployment system 22 for delivering the framework 46 to a vascular deployment site 92 within a vascular structure V of a patient. In particular, the deployment system 22, which may include one or more devices, may restrict movement of the expandable and collapsible framework 46 from a collapsed position, for transporting the framework 46, to an expanded, or deployed, position. When properly positioned, the expandable and collapsible framework 46 may be moved from the collapsed position to the expanded position.

The expandable and collapsible framework 46 may be made from a shape memory material and may be configured to self-expand when the framework 46 is no longer restricted from radial movement by the deployment system 22. In particular, the expandable and collapsible framework 46 may be positioned within a deployment catheter 24 and engaged with a deployment wire 34 configured to move the framework 46 between its collapsed position, in which catheter lumen walls 48 restrict radial movement of the framework 46, and its expanded position, in which the catheter lumen walls 48 no longer restrict radial movement of the framework 46. One or both of the deployment catheter 24 and deployment wire 34 may be moved relative to the other such that the expandable and collapsible framework 46 is moved distally beyond a distal tip 50 of the deployment catheter 24.

As an alternative to a self-expanding framework, the expandable and collapsible framework 46 may include an inflatable balloon 100 positioned therein and configured to move the framework 46 from the collapsed position to the expanded position during inflation in a known manner. According to such an embodiment, the deployment system 22 may include inflation and deflation means for the inflatable balloon 100. For example, an alternative deployment catheter 102 may be used that includes one lumen 104 for receiving the deployment wire 34 and the expandable and collapsible framework 46, and an inflation lumen 106 in fluid communication with an interior 108 of the inflatable balloon 100 via openings through the catheter 102. Thus, as should be appreciated, a fluid source may be used to inflate the inflatable balloon 100 via the inflation lumen 106.

In the expanded position, the expandable and collapsible framework 46 may have a number of different uses. For example, the expandable and collapsible framework 46 may be used to create an artificial blockage, or occlusion, within the vascular structure V. According to the embodiment utilizing an inflatable balloon 100, the inflatable balloon 100, received within the expandable and collapsible framework 46, may restrict blood flow downstream from the framework 46. Alternatively, a flow restriction membrane 120 may be supported on the expandable and collapsible framework 46 and, when the framework 46 is in the expanded position, may restrict blood flow beyond the framework 46. Yet alternatively, as shown in FIG. 11, embolic devices 130, such as embolic coils 134, may be packed within the expandable and collapsible framework 46, in its expanded position, to restrict or reduce blood flow.

Such an embolization procedure may be useful to treat several conditions, including, for example, aneurysms, hemorrhages, and lesions or growths. In addition, an embolization procedure may be used to isolate a treatment area from general circulation. For example, if a toxic agent, such as a chemotherapeutic agent, is delivered to a specific treatment site, it may be desirable to prevent circulation of the toxic agent downstream from the treatment area. As such, one or more artificial blockages may be created to effectively isolate the treatment area. The expandable and collapsible framework 46 may be detached from the deployment system 22 or may remain attached to the deployment system 22, depending on the particular embodiment used and the particular procedure being performed.

The expandable and collapsible framework 46 may also be used as a stent. For example, the expandable and collapsible framework 46 may be deployed at the vascular deployment site 92 to reinforce, repair, or otherwise provide support for the vascular structure V, or other body lumen. For example, when a patient suffers from atherosclerosis, the expandable and collapsible framework 46, in the expanded position, may be placed in a coronary or a peripheral artery at a location where the artery is weakened or damaged. The expandable and collapsible framework 46, once in place and in the expanded position, may reinforce that portion of the artery, thereby restoring normal blood flow through the vessel.

An alternative expandable and collapsible framework 146 having a semispherical shape, as shown in FIG. 13, may be positioned within the vascular structure V to filter clots from the blood flow. For example, the expandable and collapsible framework 146, which may be delivered and removed from the vascular deployment site 92, as described herein, may be positioned downstream from a clot 150, such as during a clot removal procedure. In the expanded position, the expandable and collapsible framework 146 may exert sufficient radial force against the vascular walls 140, 152 to maintain a desired position of the framework 46. As portions of the clot 150 are broken down or removed from the vascular structure walls 140, 152, the expandable and collapsible framework 146, which may include a flow restriction membrane 120 supported thereon, may capture clots 150 for removal from the vascular structure V.

The endovascular medical system including the expandable and collapsible framework described herein provides a multi-purpose device that is reliable and effective and may be used in a variety of different endovascular procedures. By utilizing a framework having the link sets described herein, the expandable and collapsible framework is capable of maintaining a desired shape in both its collapsed and expanded positions. As such, the expandable and collapsible framework may provide a structure having a controlled and predictable expansion that may be used reliably and effectively for a variety of endovascular procedures, as described herein.

It should be understood that the above description is intended for illustrative purposes only, and is not intended to limit the scope of the present disclosure in any way. Thus, those skilled in the art will appreciate that other aspects of the disclosure can be obtained from a study of the drawings, the disclosure and the appended claims.

What is claimed is:

1. An endovascular medical system, comprising:
an expandable and collapsible framework including a plurality of link sets joined together at hubs, wherein each of the link sets includes a plurality of scissors linkages connected in series, wherein the expandable and collapsible framework is movable between an expanded position in which each of the scissors linkages are pivoted to a closed position and a collapsed position in which each of the scissors linkages are pivoted to an open position; and
a deployment system for advancing the framework to a vascular deployment site within a vascular structure, wherein the deployment system restricts movement of the framework from the collapsed position to the expanded position during advancement to the vascular deployment site.

2. The endovascular medical system of claim 1, wherein the deployment system includes a deployment wire and a deployment catheter, wherein the deployment wire and the expandable and collapsible framework are axially movable within the deployment catheter.

3. The endovascular medical system of claim 1, wherein the expandable and collapsible framework has a spherical shape in both of the expanded and collapsed positions.

4. The endovascular medical system of claim 3, further including a flow restriction membrane supported on the expandable and collapsible framework.

5. The endovascular medical system of claim 3, further including a plurality of embolic devices received within the expandable and collapsible framework.

6. The endovascular medical system of claim 1, wherein the expandable and collapsible framework includes a shape memory material.

7. The endovascular medical system of claim 1, further including an inflatable balloon positioned within the expandable and collapsible framework and configured to move the expandable and collapsible framework from the collapsed position to the expanded position.

8. The endovascular medical system of claim 1, further including a retraction hook extending outwardly from the expandable and collapsible framework, wherein the endovascular medical system includes a retraction device having a retraction member shaped to engage the refraction hook.

9. The endovascular medical system of claim 1, wherein the expandable and collapsible framework has a semispherical shape in both of the expanded and collapsed positions.

10. The endovascular medical system of claim 1, wherein each of the link sets defines a continuous ring.

11. A method of performing a percutaneous endovascular procedure using an endovascular medical system, the endovascular medical system including an expandable and collapsible framework including a plurality of link sets joined together at hubs, wherein each of the link sets includes a plurality of scissors linkages connected in series, wherein the expandable and collapsible framework is movable between an expanded position in which each of the scissors linkages are pivoted to a closed position and a collapsed position in which each of the scissors linkages are pivoted to an open position, the method comprising steps of:
advancing the expandable and collapsible framework to a vascular deployment site within a vascular structure in the collapsed position, including restricting movement of the expandable and collapsible framework from the collapsed position to the expanded position using a deployment system;
reconfiguring the deployment system to allow movement of the expandable and collapsible framework from the collapsed position to the expanded position; and
moving the expandable and collapsible framework from the collapsed position to the expanded position at the vascular deployment site by pivoting each of the scissors linkages from an open position to a closed position.

12. The method of claim 11, further including maintaining a spherical shape of the expandable and collapsible framework during the moving step.

13. The method of claim 12, further including restricting a fluid flow through the expandable and collapsible framework using a flow restriction membrane supported on the expandable and collapsible framework.

14. The method of claim 12, further including:
receiving a plurality of embolic devices within the expandable and collapsible framework; and
restricting a fluid flow through the expandable and collapsible framework using the embolic devices.

15. The method of claim 11, wherein the moving step includes self-expanding the expandable and collapsible framework from the collapsed position to the expanded position.

16. The method of claim 11, wherein the moving step includes inflating an inflatable balloon positioned within the expandable and collapsible framework to move the expandable and collapsible framework from the collapsed position to the expanded position.

17. The method of claim 11, further including retracting the expandable and collapsible framework from the vascular deployment site by:
engaging a retraction member of a refraction device with a retraction hook extending outwardly from the expandable and collapsible framework to define an engaged configuration; and
withdrawing the retraction device and the expandable and collapsible framework through the vascular structure in the engaged configuration.

18. The method of claim 11, further including maintaining a semispherical shape of the expandable and collapsible framework during the moving step.

19. The method of claim 18, further including filtering a clot from a fluid flow through the expandable and collapsible framework using the plurality of link sets.

20. The method of claim 11, further including dilating the vascular structure using the expandable and collapsible framework during the moving step.
